(19) United States
(12) Patent Application Publication
(10) Pub. No.: US 2005/0187262 A1
(43) Pub. Date: Aug. 25, 2005

(54) Compositions comprising (S)-amlodipine and an angiotensin receptor blocker and methods of their use

(76) Inventors: Donna R. Grogan, Hudson, MA (US); Larry R. Bush, Worcester, MA (US)

Correspondence Address: JONES DAY, 51 Louisiana Aveue, N.W, WASHINGTON, DC, 20001-2113, US

(21) Appl. No.: 11/033,277

(22) Filed: Jan. 12, 2005

Related U.S. Application Data
(60) Provisional application No. 60/535,488, filed on Jan. 12, 2004.
(60) Provisional application No. 60/559,014, filed on Apr. 5, 2004.
(60) Provisional application No. 60/628,926, filed on Nov. 19, 2004.

(51) Int. Cl. A61K 31/455; A61K 31/4184

(52) U.S. Cl. 514/355; 514/381

(57) ABSTRACT

A pharmaceutical composition comprising enantiomerically pure (S)-amlodipine, an ARB and optional other active agents, and methods of treating, preventing and managing cardiovascular diseases and disorders, and symptoms thereof, using the composition, are disclosed.

This application claims priority to U.S. Provisional Application Nos. 60/535,488, filed Jan. 12, 2004, 60/559,014, filed Apr. 5, 2004, and 60/628,926, filed Nov. 19, 2004, all of which are incorporated herein in their entireties.

### 1. FIELD OF THE INVENTION

This invention relates to compositions and methods for treating, preventing and managing cardiovascular diseases and disorders such as, but not limited to, hypertension and angina.

### 2. BACKGROUND OF THE INVENTION

2.1 Cardiovascular Diseases and Disorders

Numerous cardiovascular diseases and disorders exist, which differ in their etiologies, severities, and effect. See, e.g., Harrison's Principles of Internal Medicine, p. 1253-5 (15^{th} ed. 2001). For example, hypertension is a serious disease that can, over time; increase a patient's risk of stroke, aneurysm, heart failure, heart attack and kidney damage, yet hypertension itself is a generally symptomless condition. Thus, while the number of Americans who have hypertension is estimated to be more than 50 million, it is estimated that only about two thirds of those are diagnosed with the disease. The Merck Manual of Medical Information Home Ed., p. 112 (1997). Moreover, only about 75 percent of those diagnosed receive drug treatment, and only about 45 percent receive adequate treatment. Id.

While still the subject of extensive research, hypertension appears to be the product of an inherited predisposition, coupled with dietary, emotional, and environmental factors, which results in a structural adaptation of the cardiac muscle and the large blood vessels. Most patients display heightened vascular and cardiac reactions to sympathetic nervous stimulation, but the precise relationship of sympathetic nervous stimulation to the etiology of the disease is unknown. Nevertheless, hypertension results in chronic readjustment of cardiovascular hemodynamics, alteration of blood vessel walls, cardiovascular resistance and regional transmural pressures.

Pharmacologic management of hypertension is generally directed to the normalization of altered hemodynamic parameters, and many drugs and drug classes, either as monotherapy or in combination treatment, can reduce and control elevated blood pressure. However, treatment of hypertension does not always improve the morbidity and mortality of the condition, either because chronic hypertension has produced other significant and irreversible cardiovascular changes, or because present drugs have an adverse effect on some other risk factor for cardiovascular disease. Rather, current drug therapy simply provides sustained arterial pressure reduction.

An example of another cardiovascular disorder is angina pectoris, which is a highly variable, rather poorly understood clinical syndrome reflecting a myocardial ischemia. When cardiac work or myocardial oxygen demand exceeds the ability of the coronary arterial vascular system to supply oxygen, the resulting ischemia stimulates the sensory nerves of the heart, producing the sensation of angina characterized by episodes of precordial pressure, discomfort, or a severe, intense crushing pain which may radiate to several sites including the left shoulder and left arm. Physical activity or exertion characteristically initiates the condition, and rest or drug therapy relieves the condition. The signs and symptoms of an episode persist for a few minutes, but can be induced or exaggerated by a meal or exposure to cold air. Treatment is directed to the underlying disease, usually atherosclerosis, or to drugs which either reduce myocardial oxygen demand or improve oxygen supply. Calcium antagonists have been particularly useful in treating vasospastic angina, the angina of effort, and the unstable angina, due to the effect of the calcium channel antagonist on cardiac and vascular smooth muscle. Treatment of angina is designed to prevent coronary artery disease, to slow its progression, or to reverse it by dealing with its known cause. Drugs such as beta-blockers, nitrates, calcium antagonists and antiplatelet drugs are used for the treatment of angina.

2.2 (S)-Amlodipine

Amlodipine, which is chemically named 2-[(2-aminoethoxy)methyl]-4-(2-chlorophenyl)-1,4-dihydro-6-methyl-3,5-pyridinedicarboxylic acid 3-ethyl 5-methyl ester, is a racemic drug belonging to the class of compounds known as calcium antagonists. The besylate salt of amlodipine is sold under the trade name NORVASC® (Pfizer, Inc.), and is indicated in the United States for the treatment of hypertension, chronic stable angina and vasospastic angina.

Although amlodipine is currently used in its racemic form, the in vitro effects of enantiomers of amlodipine have been investigated. See, e.g., Arrowsmith et al., J. Med. Chem., 29: 1696-1702 (1986), and European Patent Application No. 0331315. In addition, optically pure (−) amlodipine has been found to be an effective antihypertensive agent for both systolic and diastolic hypertension. See, e.g., U.S. Pat. No. 6,291,490, which is incorporated herein in its entirety by reference. Although (S)-amlodipine is generally well tolerated, edema may develop and cause occasional discontinuation of (S) amlodipine therapies.

2.3 Angiotensin Receptor Blockers

The renin-angiotensin system is an important participant in both the short- and long-term regulation of arterial blood pressure. Factors that decrease arterial blood pressure, such as decreases in effective blood volume (caused by, for example, a low-sodium diet, diuretics, blood loss, congestive heart failure, liver cirrhosis, or nephritic syndrome) or reductions in total peripheral resistance (caused by, for example, vasodilators), activate renin release from the kidneys.

Renin is an enzyme that acts on angiotensinogen (renin substrate) to catalyze the formation of the decapeptide angiotensin I. This decapeptide is then cleaved by angiotensin converting enzyme (ACE) to yield the octapeptide angiotensin II. Angiotensin II acts via diverse, yet coordinated, mechanisms to raise arterial blood pressure.

Angiotensin II receptor blockers (ARBs) are drugs which block or reduce the action of angiotensin II. ARBs are known to prevent and reverse the effects of angiotensin II, including, but not limited to: (1) rapid pressor responses; (2) slow pressor responses; (3) stimulatory effects on the peripheral sympathetic nervous system; (4) CNS effects; (5) release of adrenal catecholamines; (6) secretion of aldosterone; (7) direct and indirect effects of angiotensin II on the kidneys; (8) growth promoting actions and (9) contraction of vascular smooth muscle. Goodman & Gilman's The Pharmacological Basis of Therapeutics, p. 752 (9^{th} ed. 1996). Several ARBs, such as candesartan, eprosartan, irbesartan, losartan, olmesartan, telmisartan and valsartan, have been approved by the U.S. Food and Drug Administration, and are indicated in the United States for the treatment of hypertension. See, e.g., Physician's Desk Reference, p. 2068 (56^{th} ed., 2002).

### 3. SUMMARY OF THE INVENTION

This invention encompasses pharmaceutical compositions comprising enantiomerically pure (S)-amlodipine, or a pharmaceutically acceptable prodrug, salt, or solvate thereof, and an angiotensin receptor blocker (ARB), or a pharmaceutically acceptable prodrug, salt or solvate thereof.

This invention also encompasses single unit dosage forms comprising enantiomerically pure (S)-amlodipine, or a pharmaceutically acceptable prodrug, salt or solvate thereof and an ARB, or a pharmaceutically acceptable prodrug, salt or solvate thereof. Particular dosage forms are suitable for routes of administration including oral, mucosal, rectal and parenteral administration.

Another embodiment of the invention encompasses methods of treating, preventing, or managing a cardiovascular disease or disorder, or symptoms thereof, which comprise administering to a patient (e.g., a mammal such as a human) in need of such treatment, prevention or management a therapeutically or prophylactically effective amount of enantiomerically pure (S)-amlodipine, or a pharmaceutically acceptable prodrug, salt or solvate thereof, and an ARB, or a pharmaceutically acceptable prodrug, salt or solvate thereof. (S)-amlodipine and the ARB may be administered sequentially or simultaneously by the same or by different routes of administration.

In one embodiment, the invention relates to a method of treating or preventing a cardiovascular disease or disorder comprising administering to a patient in need of such treatment a therapeutically or prophylactically effective amount of enantiomerically pure (S)-amlodipine, or a pharmaceutically acceptable prodrug, salt or solvate thereof, and an ARB, or a pharmaceutically acceptable prodrug, salt or solvate thereof, wherein the ARB is losartan, candesartan, eprosartan, telmisartan, irbesartan, pratosartan, valsartan or olmesartan.

In another embodiment, the invention relates to a method of treating or preventing hypertension or angina comprising administering to a patient in need of such treatment a therapeutically or prophylactically effective amount of enantiomerically pure (S)-amlodipine, or a pharmaceutically acceptable prodrug, salt or solvate thereof, and an ARB, or a pharmaceutically acceptable prodrug, salt or solvate thereof, wherein the ARB is losartan, candesartan, eprosartan, telmisartan, irbesartan, pratosartan, valsartan or olmesartan.

In certain embodiments, one of more additional active agents may be used in addition to (S)-amlodipine and an ARB. Thus, this invention also encompasses a method of treating or preventing a cardiovascular disease or disorder comprising administering to a patient in need of such treatment or prevention a therapeutically or prophylactically effective amount of: 1) enantiomerically pure (S)-amlodipine, or a pharmaceutically acceptable prodrug, salt or solvate thereof; 2) an ARB, wherein the ARB is lorsartan, candesartan, eprosartan, telmisartan, irbesartan, pratosartan, valsartan or olmesartan; and a diuretic agent.

In a further embodiment, the invention relates to a pharmaceutical composition comprising enantiomerically pure (S)-amlodipine, or a pharmaceutically acceptable prodrug, salt or solvate thereof, and an ARB, or a pharmaceutically acceptable prodrug, salt or solvate thereof, wherein the ARB is losartan, candesartan, eprosartan, telmisartan, irbesartan, pratosartan, valsartan or olmesartan.

In a further embodiment, the invention relates to a single unit dosage form comprising enantiomerically pure (S)-amlodipine, or a pharmaceutically acceptable prodrug, salt or solvate thereof, and an ARB, or a pharmaceutically acceptable prodrug, salt or solvate thereof, wherein the ARB is losartan, candesartan, eprosartan, telmisartan, irbesartan, pratosartan, valsartan or olmesartan.

### 4. BRIEF DESCRIPTION OF FIGURES

FIG. 1A illustrates the effects of co-administration of (S)-amlodipine malate and each of telmisartan, irbesartan, olmesartan, and eprosartan.

FIG. 1B illustrates the effects of co-administration of (S)-amlodipine malate and each of losartan, valsartan, and candersartan.

### 5. DETAILED DESCRIPTION OF THE INVENTION

This invention is based, in part, on a realization that cardiovascular diseases and disorders can be treated, prevented and managed using a combination of enantiomerically pure (S)-amlodipine and certain ARBs. Without being limited by a particular theory, it is believed that relatively low doses of ARBs increase the vascular sensitivity to (S)-amlodipine. Therefore, it is further believed that, when combined with ARBs, a lower dose of (S)-amlodipine, as compared to the conventionally used dose, can be used for the treatment, prevention and management of cardiovascular diseases and disorders, which in turn can lower the incidence of side effects associated with (S) amlodipine. Furthermore, combination of two or more drugs can provide beneficial characteristics in terms of convenience and patient compliance. Accordingly, this invention encompasses various methods of treatment, as well as pharmaceutical compositions and single unit dosage forms comprising (S)-amlodipine and an ARB.

As used herein, and unless otherwise specified, the term “enantiomerically pure” means a composition that comprises one enantiomer of a compound and is substantially free of the opposite enantiomer of the compound. The term “substantially free” means a compound comprises greater than 80 percent by weight of one enantiomer of the compound and less than about 20 percent by weight of the opposite enantiomer of the compound, preferably greater than about 90 percent by weight of one enantiomer of the compound and less than about 10 percent by weight of the opposite enantiomer of the compound, and more preferably greater than about 95 percent by weight of one enantiomer of the compound and less than about 5 percent by weight of the opposite enantiomer of the compound, and even more preferably greater than about 97 percent by weight of one enantiomer of the compound and less than about 3 percent by weight of the opposite enantiomer of the compound; even more preferably greater than about 99 percent by weight of one enantiomer of the compound and less than about 1 percent by weight of the opposite enantiomer of the compound. For example, enantiomerically pure (S)-amlodipine in one embodiment comprises at least about 90 percent by weight (S)-amlodipine and less than about 10 percent by weight (R)-amlodipine. In another embodiment, enantiomerically pure (S)-amlodipine comprises at least about 95 percent by weight (S)-amlodipine and less than about 5 percent by weight of (R)-amlodipine. In a further embodiment, enantiomerically pure (S)-amlodipine comprises at least about 97 percent by weight (S)-amlodipine and less than about 3 percent by weight of (R)-amlodipine. In yet another embodiment, enantiomerically pure (S)-amlodipine comprises at least about 99 percent by weight (S)-amlodipine and less than about 1 percent by weight of (R)-amlodipine.

S-amlodipine and salts thereof may be prepared by methods including, but not limited to, those described herein and in U.S. Pat. No. 6,291,490; U.S. Pat. No. 6,608,206; U.S. Pat. No. 6,646,131 and published U.S. applications U.S. 2003/130321A1 and U.S. 2003/176706A1, all of which are incorporated herein in their entirety by reference.

As used herein, and unless otherwise specified, the term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties. As used herein, the term “prodrug,” when used in connection with enantiomerically pure (S)-amlodipine, does not encompass racemic amlodipine or prodrugs of racemic amlodipine.

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, and unless otherwise specified, the term “pharmaceutically acceptable salt” refers to salts prepared from pharmaceutically acceptable non-toxic acids, including inorganic acids and organic acids. Suitable non-toxic acids include inorganic and organic acids such as, but not limited to, acetic, alginic, anthranilic, aspartic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleate, maleic, malic, mandelic, methanesulfonic, mucic, nicotinic, nitric, pamoic, pantothenic, phenylacetic, propionic, phosphoric, salicylic, stearic, succinic, sulfanilic, sulfuric, tartaric acid, p-toluenesulfonic and the like. Specific salts are prepared from hydrochloric, hydrobromic, phosphoric, and sulfuric acids. In one embodiment, specific salts of (S)-amlodipine include, but are not limited to, the malate, besylate, maleate, camsylate, tosylate, nicotinate, L-hemitartrate, and D-hemitartrate salts. In another embodiment, specific salt of (S)-amlodipine is not malate salt.

As used herein, and unless otherwise specified, the term “solvate” means a molecular or ionic complex formed by one or more molecules or ions of the solvent with one or more molecules or ions of solute. Where the solvent is water, the solvate is a hydrate.

Angiotensin receptor blockers that can be used in methods and composition of the invention are well known in the art. See, e.g., Physician's Desk Reference (56^{th} ed. 2002). In addition, the ARB activity of as yet untested compounds can be readily identified using any methods well-known in the art, including, but not limited to, pressor responses attenuation assays and selective binding assays using an angiotensin receptor (e.g., AT₁ receptor). Specific ARBs that can be used in the invention include, but are not limited to, candesartan, eprosartan, telmisartan, irbesartan, pratosartan, valsartan, olmesartan, losartan, tasosartan, embusartan, GA-01 13, KRH-594 and UR-7247, or pharmaceutically acceptable prodrugs, salts, or solvates thereof. The ARBs can be racemic or enantiomerically pure.

Candesartan is chemically named 2-ethoxy-1-[[2′-(1H-tetrazol-5-yl)[1,1′-biphenyl]-4-yl]methyl]-1H-benzimidazole-7-carboxylic acid, and candesartan cilexetil is sold in the United States under the trade name ATACAND® (AstraZeneca). Candesartan may be prepared by methods disclosed and described in U.S. Pat. No. 5,196,444; U.S. Pat. No. 5,534,534; U.S. Pat. No. 5,703,110; and U.S. Pat. No. 5,705,517, all of which are incorporated herein by reference.

Eprosartan is chemically named (E)-α-[[2-butyl-1-[(4-carboxyphenyl)methyl]-1H-imidazol-5-yl]methylene]-2-thiophenepropanoic acid, and eprosartan mesylate is sold in the United States under the trade name TEVETAN® (Biovail). TEVETAN SB®, which reportedly exhibits a better bioavailability, is also available from Biovail. Eprosartan may be prepared by methods disclosed and described in U.S. Pat. No. 5,185,351 and U.S. Pat. No. 5,656,660, all of which are incorporated herein by reference.

Telmisartan is chemically named 4′-[(1,4′-dimethyl-2′-propyl[2,6′-bi-1H-benzimidazol]-1′-yl)methyl]-[1,1′-biphenyl]-2-carboxylic acid, and is sold in the United States under the trade name MICARDIS® (Boehringer Ingelheim). Telmisartan may be prepared by methods disclosed and described in U.S. Pat. No. 5,591,762 and U.S. Pat. No. 6,358,762, all of which are incorporated herein by reference.

Irbesartan is chemically named 2-butyl-3-[[2′-(1H-tetrazol-5-yl)[1,1′-bephenyl]-4-yl]methyl]-1,3-diazaspiro[4,4]non-1-en-4-one, and is sold in the United States under the trade name AVAPRO® (Bristol-Myers Squibb). Irbesartan may be prepared by methods disclosed and described in U.S. Pat. No. 5,270,317; U.S. Pat. No. 5,994,348; and U.S. Pat. No. 6,342,247, all of which are incorporated herein by reference.

Losartan is chemically named 2-butyl-4-chloro-1-[[2′-(1H-tetrazol-5-yl)[1,1′-biphenyl]-4-yl]methyl]-1H-imidazole-5-methanol, and losartan potassium is sold in the United States under the trade name COZAAR® (Merck). Losartan may be prepared by methods disclosed and described in U.S. Pat. No. 5,608,075; U.S. Pat. No. 5,138,069; U.S. Pat. No. 5,153,197; and U.S. Pat. No. 5,210,079, all of which are incorporated herein by reference.

Pratosartan is chemically named 2-propyl-3-[[2′-(1H-terazol-5-yl)biphenyl-4-yl]methyl]-5,6,7,8-tetrahydrocycloheptaimidazol-4(3H)-one, and is commercially obtainable from Kotobuki.

Valsartan is chemically named as N-(1-oxopentyl)-N-[[2′-(1H-tetrazol-5-yl)[1,1′-biphenyl]-4-yl]methyl]-L-valine, and is sold in the United States under the trade name DIOVAN® (Novartis). Valsartan may be prepared by methods disclosed and described in U.S. Pat. No. 5,399,578 and U.S. Pat. No. 6,294,197, all of which are incorporated herein by reference.

Olmesartan is chemically named as 2,3-dihydroxy-2-butenyl-4-(1-hydroxy-1-methylethyl)-2-propyl-1-[p-(o-1H-terazol-5-yl-phenyl)-benzyl]-immidazole-5-carboxylate, cyclic 2,3 carbonate, and olmesartan medoxomil is sold in the United States under the trade name BENICAR® (Sankyo Pharmaceuticals, Inc.). Olmesartan may be prepared by methods disclosed and described in U.S. Pat. No. 5,616,599, incorporated herein by reference.

In certain embodiments of the invention, the ARB is not losartan. In other embodiments, wherein the ARB is losartan, the (S)-amlodipine does not exist as a besylate or maleate salt. In another embodiment, the ARB is eprosartan, in particular, TEVATAN® or TEVATAN SB®. In another embodiment, the ARB is valsartan. In another embodiment, the ARB is irbesartan. In another embodiment, the ARB is telmisartan. In another embodiment, the ARB is cadesartan, pratosartan, or olmesartan.

5.1 Methods of Treatment, Prevention or Management

This invention encompasses methods of treating, preventing and managing cardiovascular diseases and disorders comprising administering to a patient (e.g., a mammal such as a human) in need of such treatment, prevention or management a therapeutically or prophylactically effective amount of enantiomerically pure (S)-amlodipine, or a pharmaceutically acceptable prodrug, salt or solvate thereof, and an ARB, or a pharmaceutically acceptable prodrug, salt or solvate thereof.

The ARB and enantiomerically pure (S)-amlodipine may be administered sequentially or simultaneously by the same or different routes of administration. In one embodiment, the ARB and enantiomerically pure (S)-amlodipine are simultaneously administered in a pharmaceutical composition.

This invention also encompasses methods of treating, preventing and managing cardiovascular diseases and disorders in a patient (e.g., a mammal such as a human) in need of such treatment, prevention or management comprising administering pharmaceutical compositions comprising enantiomerically pure (S)-amlodipine, or a pharmaceutically acceptable prodrug, salt or solvate thereof, and an angiotensin receptor blocker, or a pharmaceutically acceptable prodrug, salt or solvate thereof, wherein the angiotensin receptor blocker is losartan, candesartan, eprosartan, telmisartan, irbesartan, pratosartan, valsartan, or olmesartan. In one embodiment, the angiotensin receptor blocker is candesartan, olmesartan, or pratosartan. In another embodiment, the ARB is eprosartan, in particular, TEVATAN® or TEVATAN SB®. In another embodiment, the ARB is valsartan. In another embodiment, the ARB is irbesartan. In another embodiment, the ARB is telmisartan.

Cardiovascular diseases or disorders that can be treated, prevented or managed using methods of this invention include, but are not limited to, acute or chronic renal failure, angina pectoris, arterial spasm, cardiac arrhythmias, cardiac hypertrophy, cerebral ischemia, congestive heart failure, coronary myocardial infarction, diabetic nephropathy, hypertension, including but not limited to chronic, systolic and diastolic hypertension, ischemia reperfusion injury, ischemic myocardial necrosis, Raynaud's phenomenon, renal impairment, stroke and others well-known in the art. See, e.g., Harrison's Principles of Internal Medicine, p. 1253-5 (15^{th} ed. 2001). Some symptoms of cardiovascular diseases or disorders that can be treated, prevented or managed using methods of this invention include, but are not limited to, asthma, bronchospasm, cognitive disorders such as, but not limited to, dementia and age-associated memory impairment, and migraines. In one embodiment, the disease or disorder is hypertension. In another embodiment, the disease or disorder is angina.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder.

As used herein, and unless otherwise specified, the terms “prevent,” “preventing” and “prevention” refer to the prevention of the onset, recurrence or spread of a disease or disorder, or of one or more symptoms thereof.

As used herein, and unless otherwise specified, the terms “manage,” “managing” and “management” refer to preventing or slowing the progression, spread or worsening of a disease or disorder, or of one or more symptoms thereof. Often, the beneficial effects that a subject derives from a prophylactic or therapeutic agent do not result in a cure of the disease or disorder.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

In one embodiment, the enantiomerically pure (S)-amlodipine comprises at least about 80 percent, 85 percent, 90 percent, 95 percent, 97 percent, or 99 percent by weight of the total amlodipine used. Enantiomerically pure (S)-amlodipine is preferably administered in an amount of from about 0.01 mg to about 100 mg per day, from about 0.1 mg to about 50 mg per day, from about 0.5 mg to about 20 mg per day, or from about 1 mg to about 10 mg per day.

Angiotensin receptor inhibitors that can be used in connection with methods of this invention include, but are not limited to, candesartan, eprosartan, telmisartan, irbesartan, pratosartan, valsartan, olmesartan, losartan, tasosartan, embusartan, GA-0113, KRH-594 and UR-7247, or pharmaceutically acceptable prodrugs, salts, or solvates thereof. The ARBs can be racemic or enantiomerically pure.

In certain embodiments, the ARB is not losartan. In other embodiments, wherein the ARB is losartan, the (S)-amlodipine does not exist as a besylate or maleate salt. In yet other embodiments, wherein the ARB is losartan and the (S)-amlodipine exists as a besylate or maleate salt, the disease or disorder to be treated, prevented or managed is not hypertension. In other embodiments, specific salt form of (S)-amlodipine is not malate salt.

In another embodiment, the ARB is candesartan, eprosartan, telmisartan, irbesartan, pratosartan, valsartan, tasosartan, or olmesartan. In a further embodiment, the ARB is candesartan, pratosartan, or olmesartan. In another embodiment, the ARB is irbesartan, telmisartan, or olmesartan. In yet another embodiment, the ARB is eprosartan. In another embodiment, the ARB is valsartan. In another embodiment, the ARB is irbesartan. In another embodiment, the ARB is telmisartan.

In another aspect of the invention, the ARB is losartan. In a further aspect, the ARB is olmesartan. In another embodiment, the ARB is telmisartan. In another embodiment, the ARB is eprosartan, in particular, TEVATAN® or TEVATAN SB®.

Candesartan is preferably used in an amount of from about 0.1 mg to about 100 mg per day, from about 0.5 mg to about 70 mg per day, or from about 1 mg to about 50-mg per day. Eprosartan is preferably used in an amount of from about 10 mg to about 1500 mg per day, from about 50 mg to about 1000 mg per day, or from about 100 mg to about 700 mg per day. Telmisartan is preferably used in an amount of from about 0.1 mg to about 300 mg per day, from about 1 mg to about 200 mg per day, or from about 10 mg to about 100 mg per day. Irbesartan is preferably used in an amount of from about 10 mg to about 1500 mg per day, from about 25 mg to about 1000 mg per day, or from about 50 mg to about 500 mg per day. Tasosartan is preferably used in an amount of from about 10 mg to about 1500 mg per day, from about 50 mg to about 1000 mg per day, or from about 100 mg to about 700 mg per day.

Valsartan is preferably used in an amount of from about 1 mg to about 1000 mg per day, from about 10 mg to about 700 mg per day, or from about 30 mg to about 500 mg per day. Olmesartan is preferably used in an amount of from about 0.1 mg to about 100 mg per day, from about 1 mg to about 70 mg per day, or from about 3 mg to about 50 mg per day. Losartan is preferably used in an amount of from about 0.1 mg to about 500 mg per day, from about 1 mg to about 200 mg per day, or from about 25 mg to about 100 mg per day. Pratosartan is preferably used in an amount of from about 0.1 mg to about 2000 mg per day, from about 1 mg to about 1000 mg per day, from about 10 mg to about 700 mg per day, or from about 30 mg to about 500 mg per day.

The selected dosage level and frequency of administration of the pharmaceutical compositions of the invention will depend upon a variety of factors including the route of administration, the time of administration, the rate of excretion of the therapeutic agents, the duration of the treatment, other drugs, compounds and/or materials used in the patient, the age, sex, weight, condition, general health and prior medical history of the patient being treated, and like factors well known in the medical arts. For example, the dosage regimen is likely to vary with pregnant women, nursing mothers and children relative to healthy adults. A physician having ordinary skill in the art can readily determine and prescribe the therapeutically effective amount of the pharmaceutical composition required.

In methods of this invention, one or more additional active agents may be used in addition to (S)-amlodipine, or a pharmaceutically acceptable salt, solvate or prodrug thereof, and an ARB. Particular active agents are diuretic agents. Accordingly, this invention also encompasses a method of treating, preventing or managing a cadiovascular disease or disorder comprising administering to a patient in need of such treatment, prevention or management a therapeutically or prophylactically effective amount of: 1) enantiomerically pure (S)-amlodipine, or a pharmaceutically acceptable prodrug, salt or solvate thereof; 2) an ARB, wherein the ARB is lorsartan, candesartan, eprosartan, telmisartan, irbesartan, pratosartan, valsartan or olmesartan; and a diuretic agent. In certain methods of this invention, (S)-amlodipine, or a pharmaceutically acceptable prodrug, salt or solvate thereof, may be used in combination with diuretic agents in the absence of ARBs.

Examples of diuretic agents that can be used in connection with methods of this invention include, but are not limited to: carbonic anhydrase inhibitors such as acetazolamide, dichlorphenamide and methazolamide; osmotic diuretics such as glycerin, isosorbide, mannitol and urea; Na⁺—K⁺-2Cl⁻ symport inhibitors such as furosemide, bumetanide, azosemide, piretanide, ethacrynic acid, muzolimine, torsemide and tripamide; Na⁺—Cl⁻ symport inhibitors such as bendroflumethiazide, chlorothiazide, hydrochlorothiazide, dihydrochlorothiazide, hydroflumethiazide, methylchlorthiazide, polythiazide, trichlormethiazide, chlorthalidone, indapamide, metolazone and quinethazone; renal epithelial Na⁺ channels inhibitors such as amiloride and triameterene; aldosterone antagonists such as spironolactone; and other diuretic agents such as conivaptan, mozavaptan, and eplerenone.

5.2 Pharmaceutical Compositions

One embodiment of this invention is directed to a pharmaceutical composition comprising enantiomerically pure (S)-amlodipine, or a pharmaceutically acceptable prodrug, salt or solvate thereof, and an ARB, or a pharmaceutically acceptable prodrug, salt or solvate thereof. Pharmaceutical compositions of this invention can also contain one or more additional active agents, such as, but not limited to, diuretics. In certain embodiments, compositions of this invention may contain (S) amlodipine, or a pharmaceutically acceptable prodrug, salt or solvate thereof, and one or more diuretic agents, in the absence of ARBs.

Examples of ARBs that can be used in compositions of this invention include, but are not limited to, candesartan, eprosartan, telmisartan, irbesartan, pratosartan, valsartan, olmesartan, losartan, tasosartan, embusartan, GA-0113, KRH-594 and UR-7247, or pharmaceutically acceptable prodrugs, salts, or solvates thereof.

In one embodiment, the ARB is not losartan. In other embodiments, wherein the ARB is losartan, the (S)-amlodipine is not in a besylate or maleate salt form. In other embodiments, specific salt form of (S)-amlodipine is not malate salt.

In another embodiment, the ARB is candesartan, eprosartan, telmisartan, irbesartan, pratosartan, valsartan or olmesartan. In yet another embodiment, the ARB is cadesartan, pratosartan, or olmesartan. In still another embodiment, the ARB is irbesartan, telmisartan, or olmesartan. In a further embodiment, the ARB is irbesartan. In another embodiment, the ARB is losartan. In another embodiment, the ARB is eprosartan, in particular, TEVATAN® or TEVATAN SB®. In another embodiment, the ARB is valsartan. In another embodiment, the ARB is telmisartan.

Candesartan is preferably used in an amount of from about 0.1 mg to about 100 mg, from about 0.5 mg to about 70 mg, or from about 1 mg to about 50 mg. Eprosartan is preferably used in an amount of from about 10 mg to about 1500 mg, from about 50 mg to about 1000 mg, or from about 100 mg to about 700 mg. Telmisartan is preferably used in an amount of from about 0.1 mg to about 300 mg, from about 1 mg to about 200 mg, or from about 10 mg to about 100 mg. Irbesartan is preferably used in an amount of from about 10 mg to about 1500 mg, from about 25 mg to about 1000 mg, or from about 50 mg to about 500 mg. Tasosartan is preferably used in an amount of from about 10 mg to about 1500 mg, from about 50 mg to about 1000 mg, or from about 100 mg to about 700 mg.

Valsartan is preferably used in an amount of from about 1 mg to about 1000 mg, from about 10 mg to about 700 mg, or from about 30 mg to about 500 mg. Olmesartan is preferably used in an amount of from about 0.1 mg to about 100 mg, from about 1 mg to about 70 mg, or from about 3 mg to about 50 mg. Losartan is preferably used in an amount of from about 0.1 mg to about 500 mg per day, from about 1 mg to about 200 mg per day, or from about 25 mg to about 100 mg per day. Pratosartan is preferably used in an amount of from about 0.1 mg to about 2000 mg per day, from about 1 mg to about 1000 mg per day, from about 10 mg to about 700 mg per day, or from about 30 mg to about 500 mg per day.

In another embodiment, the amount of (S)-amlodipine is from about 0.01 mg to about 100 mg. In more specific embodiments, the amount of (S)-amlodipine is from about 0.1 mg to about 50 mg, from about 0.5 mg to about 20 mg, or from about 1 mg to about 10 mg.

The preferred amount of each of the active ingredients in all the dosage forms made in accordance with the present invention should be a therapeutically effective amount thereof, which is also a medically acceptable amount thereof. Actual dosage levels of each active ingredient in the pharmaceutical compositions of the present invention may be varied so as to obtain an amount of each that is effective to achieve the desired therapeutic response for a particular patient, pharmaceutical composition, and mode of administration, without being toxic to the patient.

Examples of diuretic agents that can be used in connection with compositions of this invention include, but are not limited to: carbonic anhydrase inhibitors such as acetazolamide, dichlorphenamide and methazolamide; osmotic diuretics such as glycerin, isosorbide, mannitol and urea; Na⁺—K⁺-2Cl⁻ symport inhibitors such as furosemide, bumetanide, azosemide, piretanide, ethacrynic acid, muzolimine, torsemide and tripamide; Na⁺—Cl⁻ symport inhibitors such as bendroflumethiazide, chlorothiazide, hydrochlorothiazide, dihydrochlorothiazide, hydroflumethiazide, methylchlorthiazide, polythiazide, trichlormethiazide, chlorthalidone, indapamide, metolazone and quinethazone; renal epithelial Na⁺ channels inhibitors such as amiloride and triameterene; aldosterone antagonists such as spironolactone; and other diuretic agents such as conivaptan, mozavaptan, and eplerenone.

The pharmaceutical compositions of the invention comprising (S)-amlodipine, or a pharmaceutically acceptable prodrug, salt, or solvate thereof, and an ARB, or a pharmaceutically acceptable prodrug, salt, or solvate thereof, may further comprise a pharmaceutically acceptable carrier. The term “pharmaceutically acceptable carrier” means one or more pharmaceutically acceptable excipients. Examples of such excipients are well known in the art and are listed in the USP (XXI)/NF (XVI), incorporated herein in its entirety by reference thereto, and include without limitation, binders, diluents, fillers, disintegrants, super disintegrants, lubricants, surfactants, antiadherents, stabilizers, and the like. The term “additives” is synonymous with the term “excipients” as used herein.

The term “pharmaceutically acceptable” is used herein to refer to those compounds, materials, compositions and/or dosage forms which are, within the scope of sound medical judgment, suitable for administration to and for use in contact with the tissues and fluids of human beings and animals without excessive toxicity, irritation, allergic response, or other problem or complication, commensurate with a reasonable medically sound benefit/risk ratio.

Further, the term “pharmaceutically acceptable” excipient is employed to mean that there are no untoward chemical or physical incompatibilities between the active ingredients and any of the excipient components of a given dosage form. For example, an untoward chemical reaction is one wherein the potency of (S)-amlodipine is detrimentally reduced or increased due to the addition of one or more excipients. Another example of an untoward chemical reaction is one wherein the taste of the dosage form becomes excessively sweet, sour or the like to the extent that the dosage form becomes unpalatable. Each excipient must be “acceptable” in the sense of being compatible with the other ingredients of the formulation and not injurious to the patient.

Physical incompatibility refers to incompatibility among the various components of the dosage form and any excipient(s) thereof. For example, the combination of the excipient(s) and the active ingredient(s) may form an excessively hygroscopic mixture or an excessively segregated mixture to the degree that the desired shape of the dosage form (e.g., tablet or troche), its stability or the like cannot be sufficiently maintained to be able to administer the dosage form in compliance with a prescribed dosage regimen as desired.

It is noted that all excipients used in the pharmaceutical compositions or dosage forms made in accordance with the present invention preferably meet or exceed the standards for pharmaceutical ingredients and combinations thereof in the USP/NF. The purpose of the USP/NF is to provide authoritative standards and specifications for materials and substances and their preparations that are used in the practice of the healing arts. The USP/NF establish titles, definitions, descriptions, and standards for identity, quality, strength, purity, packaging and labeling, and also, where practicable provide bioavailability, stability, procedures for proper handling and storage and methods for their examination and formulas for their manufacture or preparation.

The stability of a pharmaceutical product may be defined as the capability of a particular formulation, in a specific container, to remain within its physical, chemical, microbiological, therapeutic and toxicological specification, although there are exceptions, and to maintain at least about 90% of labeled potency level. Thus, for example, expiration dating is defined as the time in which the pharmaceutical product will remain stable when stored under recommended conditions.

Many factors affect the stability of a pharmaceutical product, including the stability of the therapeutic ingredient(s), the potential interaction between therapeutic and inactive ingredients and the like. Physical factors such as heat, light and moisture may initiate or accelerate chemical reactions.

This invention comprises single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as hard gelatin or soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration requires enteric coatings to protect the compounds of this invention from degradation within the gastrointestinal tract. In another example, the compounds of this invention may be administered in a liposomal formulation to shield the compounds from degradative enzymes, facilitate transport in circulatory system, and effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

5.2.1 Oral Dosage Forms

Pharmaceutical compositions of the invention that are suitable for oral administration can be presented as discrete dosage forms, such as, but are not limited to, tablets, chewable tablets, caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

Disintegrants or lubricants can be used in pharmaceutical compositions and dosage forms of the invention. Production of pharmaceutical compositions or dosage forms in accordance with the present invention may require, in addition to the therapeutic drug ingredients, excipients or additives including, but not limited to, diluents, binders, lubricants, disintegrants, colorants, flavors, sweetening agents and the like or mixtures thereof. By the incorporation of these and other additives, a variety of dosage forms (e.g., tablets, capsules, caplets, troches and the like) may be made. These include, for example, hard gelatin capsules, caplets, sugar-coated tablets, enteric-coated tablets to delay action, multiple compressed tablets, prolonged-action tablets, tablets for solution, effervescent tablets, buccal and sublingual tablets, troches and the like.

Hence, unit dose forms or dosage formulation of a pharmaceutical composition of the present invention, such as a troche, a tablet or a capsule, may be formed by combining a desired amount of each of the active ingredients with one or more pharmaceutically compatible or acceptable excipients, as described below, in pharmaceutically compatible amounts to yield a unit dose dosage formulation the desired amount of each active ingredient. The dose form or dosage formulation may be formed by methods well known in the art.

Tablets are often a preferred dosage form because of the advantages afforded both to the patient (e.g., accuracy of dosage, compactness, portability, blandness of taste as well as ease of administration) and to the manufacturer (e.g., simplicity and economy of preparation, stability as well as convenience in packaging, shipping and dispensing). Tablets are solid pharmaceutical dosage forms containing therapeutic drug substances with or without suitable additives.

Tablets are typically made by molding, by compression or by generally accepted tablet forming methods. Accordingly, compressed tablets are usually prepared by large-scale production methods while molded tablets often involve small-scale operations. For example, there are three general methods of tablet preparation: (1) the wet-granulation method; (2) the dry-granulation method; and (3) direct compression. These methods are well known to those skilled in the art. See, e.g., Remington's Pharmaceutical Sciences, 16th and 18th Eds., Mack Publishing Co., Easton, Pa. (1980 and 1990). See also U.S. Pharmacopeia XXI, U.S. Pharmacopeial Convention, Inc., Rockville, Md. (1985).

Various tablet formulations may be made in accordance with the present invention. These include tablet dosage forms such as sugar-coated tablets, film-coated tablets, enteric-coated tablets, multiple-compressed tablets, prolonged action tablets and the like. Sugar-coated tablets (SCT) are compressed tablets containing a sugar coating. Such coatings may be colored and are beneficial in covering up drug substances possessing objectionable tastes or odors and in protecting materials sensitive to oxidation. Film-coated tablets (FCT) are compressed tablets which are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in the present invention. Enteric-coated tablets (ECT) are compressed tablets coated with substances that resist dissolution in gastric fluid but disintegrate in the intestine. Enteric coating can be used for tablets containing drug substances which are inactivated or destroyed in the stomach, for those which irritate the mucosa or as a means of delayed release of the medication.

Multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, for example, U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of tablets (i.e., layered tablets and press-coated tablets) may be used, for example, in the design of prolonged-action dosage forms of the present invention.

Pharmaceutical compositions or unit dosage forms of the present invention in the form of prolonged-action tablets may comprise compressed tablets formulated to release the drug substance in a manner to provide medication over a period of time. There are a number of tablet types that include delayed-action tablets in which the release of the drug substance is prevented for an interval of time after administration or until certain physiological conditions exist. Repeat action tablets may be formed that periodically release a complete dose of the drug substance to the gastrointestinal fluids. Also, extended release tablets that continuously release increments of the contained drug substance to the gastrointestinal fluids may be formed.

In order for medicinal substances or therapeutic ingredients of the present invention, with or without excipients, to be made into solid dosage forms (e.g., tablets) with pressure, using available equipment, it is necessary that the material, either in crystalline or powdered form, possess a number of physical characteristics. These characteristics can include, for example, the ability to flow freely, as a powder to cohere upon compaction, and to be easily released from tooling. Since most materials have none or only some of these properties, methods of tablet formulation and preparation have been developed to impart these desirable characteristics to the material which is to be compressed into a tablet or similar dosage form.

As noted, in addition to the drugs or therapeutic ingredients, tablets and similar dosage forms may contain a number of materials referred to as excipients or additives. These additives are classified according to the role they play in the formulation of the dosage form such as a tablet, a caplet, a capsule, a troche or the like. One group of additives include, but are not limited to, binders, diluents (fillers), disintegrants, lubricants, and surfactants. In one embodiment, the diluent, binder, disintegrant, and lubricant are not the same.

A binder is used to provide a free-flowing powder from the mix of tablet ingredients so that the material will flow when used on a tablet machine. The binder also provides a cohesiveness to the tablet. Too little binder will give flow problems and yield tablets that do not maintain their integrity, while too much can adversely affect the release (dissolution rate) of the drugs or active ingredients from the tablet. Thus, a sufficient amount of binder should be incorporated into the tablet to provide a free-flowing mix of the tablet ingredients without adversely affecting the dissolution rate of the drug ingredients from the tablet. With lower dose tablets, the need for good compressibility can be eliminated to a certain extent by the use of suitable diluting excipients called compression aids. The amount of binder used varies upon the type of formulation and mode of administration, and is readily discernible to those of ordinary skill in the art.

Binders suitable for use with dosage formulations made in accordance with the present invention include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (povidone), methyl cellulose, pre-gelatinized starch, hydroxypropyl methyl cellulose, (e.g., Nos. 2208, 2906, 2910), microcrystalline cellulose or mixtures thereof. Suitable forms of microcrystalline cellulose can include, for example, the materials sold as AVICEL-PH-101, AVICEL-PH-103 and AVICEL-PH-105 (available from FMC Corporation, American Viscose Division, Avicel Sales, Marcus Hook, Pa., U.S.A.).

Fillers or diluents are used to give the powder (e.g., in the tablet or capsule) bulk so that an acceptable size tablet, capsule or other desirable dosage form is produced. Typically, therapeutic ingredients are formed in a convenient dosage form of suitable size by the incorporation of a diluent therewith. As with the binder, binding of the drug(s) to the filler may occur and affect bioavailability. Consequently, a sufficient amount of filler should be used to achieve a desired dilution ratio without detrimentally affecting release of the drug ingredients from the dosage form containing the filler. Further, a filler that is physically and chemically compatible with the therapeutic ingredient(s) of the dosage form should be used. The amount of filler used varies upon the type of formulation and mode of administration, and is readily discernible to those of ordinary skill in the art. Examples of fillers include, but are not limited to, lactose, glucose, sucrose, fructose, talc, calcium carbonate (e.g., granules or powder), microcrystalline cellulose, powdered cellulose, dextrates, kaolin, mannitol, silicic acid, sorbitol, starch, pre-gelatinized starch, or mixtures thereof.

Disintegrants are used to cause the dose form (e.g., tablet) to disintegrate when exposed to an aqueous environment. Too much of a disintegrant will produce tablets which may disintegrate in the bottle due to atmospheric moisture. Too little may be insufficient for disintegration to occur and may thus alter the rate and extent of release of drug(s) or active ingredient(s) from the dosage form. Thus, a sufficient amount of disintegrant that is neither too little nor too much to detrimentally alter the release of the drug ingredients should be used to form the dosage forms made according to the present invention. The amount of disintegrant used varies based upon the type of formulation and mode of administration, and is readily discernible to the skilled artisan. Examples of disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof.

When a dose form that dissolves fairly rapidly upon administration to the subject, e.g., in the subject's stomach is desired, a super disintegrant can be used, such as, but not limited to, croscarmellose sodium or sodium starch glycolate. The term “super disintegrant,” as used herein, means a disintegrant that results in rapid disintegration of drug or active ingredient in the stomach after oral administration. Use of a super disintegrant can facilitate the rapid absorption of drug or active ingredient(s) which may result in a more rapid onset of action.

With regard to tablet dosage forms, adhesion of the dosage form ingredients to the punches of manufacturing machines (e.g., tableting machine) must be avoided. For example, when drug accumulates on the punch surfaces, tablet surfaces may become pitted and therefore unacceptable. Also, sticking of drug or excipients in this way requires unnecessarily high ejection forces when removing the tablet from the die. Excessive ejection forces may lead to a high breakage rate and increase the cost of production not to mention excessive wear and tear on the dies. In practice, it is possible to reduce sticking by wet-massing or by the use of high levels of lubricants, e.g., magnesium stearate. In addition, selection of drug salts and/or excipients with good anti-adhesion properties can also minimize these problems.

As noted, the lubricant is used to enhance the flow of the tableting powder mix to the tablet machine and to prevent sticking of the tablet in the die after the tablet is compressed. Too little lubricant will not permit satisfactory tablets to be made and too much may produce a tablet with a water-impervious hydrophobic coating, which can form because lubricants are usually hydrophobic materials such as stearic acid, magnesium stearate, calcium stearate and the like. Further, a water-impervious hydrophobic coating can inhibit disintegration of the tablet and dissolution of the drug ingredient(s). Thus, a sufficient amount of lubricant should be used that readily allows release of the compressed tablet from the die without forming a water-impervious hydrophobic coating that detrimentally interferes with the desired disintegration and/or dissolution of the drug ingredient(s).

Examples of suitable lubricants for use with the present invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, or mixtures thereof. Additional lubricants include, for example, a syloid silica gel (AEROSIL 200, manufactured by W.R. Grace Co. of Baltimore Md.), a coagulated aerosol of synthetic silica (marketed by Deaussa Co. of Plano, Tex.), CAB-O-SIL (a pyrogenic silicon dioxide product sold by Cabot Co. of Boston, Mass.) or mixtures thereof.

Surfactants are used in dosage forms to improve the wetting characteristics and/or to enhance dissolution, and are particularly useful in pharmaceutical compositions or dosage forms containing poorly soluble or insoluble drug(s) or active ingredients. Examples of surfactants include, but are not limited to, polyoxyethylene sorbitan fatty acid esters, such as those commercially available as TWEENs (e.g. Tween 20 and Tween 80), polyethylene glycols, polyoxyethylene stearates, polyvinyl alcohol, polyvinylpyrrolidone, poly(oxyethylene)/poly(oxypropylene) block co-polyers such as poloxamers (e.g., commercially available as PLURONICs), and tetrafunctional block copolymers derived from sequential addition of propylene oxide and ethylene oxide to ethylenediamine, such as polyxamines (e.g., commercially as TETRONICs (BASF)), dextran, lecithin, dialkylesters of sodium sulfosuccinic acid, such as Aerosol OT, sodium lauryl sulfate, alkyl aryl polyether sulfonates or alcohols, such as TRITON X-200 or tyloxapol, p-isononylphenoxypoly (glycidol) (e.g. Olin-IOG or Surfactant 10-G (Olin Chemicals), or mixtures thereof. Other pharmaceutically acceptable surfactants are well known in the art, and are described in detail in the Handbook of Pharmaceutical Excipients, 4^{th} Ed., Pharmaceutical Press, London, IJK and American Pharmaceutical Association, Washington, D.C. (2003).

Other classes of additives for use with the pharmaceutical compositions or dosage forms of the present invention include, but are not limited to, anti-caking or antiadherent agents, antimicrobial preservatives, coating agents, colorants, desiccants, flavors and perfumes, plasticizers, viscosity increasing agents, sweeteners, buffering agents, humectants and the like.

Examples of anti-caking agents include, but are not limited to, calcium silicate, magnesium silicate, silicon dioxide, colloidal silicon dioxide, talc, or mixtures thereof.

Examples of antimicrobial preservatives include, but are not limited to, benzalkonium chloride solution, benzethonium chloride, benzoic acid, benzyl alcohol, butyl paraben, cetylpyridinium chloride, chlorobutanol, cresol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymol, or mixtures thereof.

Examples of colorants for use with the present invention include, but are not limited to, pharmaceutically acceptable dyes and lakes, caramel, red ferric oxide, yellow ferric oxide or mixtures thereof. Examples of desiccants include, but are not limited to, calcium chloride, calcium sulfate, silica gel or mixtures thereof.

Flavors that may be used include, but are not limited to, acacia, tragacanth, almond oil, anethole, anise oil, benzaldehyde, caraway, caraway oil, cardamom oil, cardamom seed, compound cardamom tincture, cherry juice, cinnamon, cinnamon oil, clove oil, cocoa, coriander oil, eriodictyon, eriodictyon fluidextract, ethyl acetate, ethyl vanillin, eucalyptus oil, fennel oil, glycyrrhiza, pure glycyrrhiza extract, glycyrrhiza fluidextract, lavender oil, lemon oil, menthol, methyl salicylate, monosodium glutamate, nutmeg oil, orange flower oil, orange flower water, orange oil, sweet orange peel tincture, compound orange spirit, peppermint, peppermint oil, peppermint spirit, pine needle oil, rose oil, stronger rose water, spearmint, spearmint oil, thymol, tolu balsam tincture, vanilla, vanilla tincture, and vanillin or mixture thereof.

Examples of sweetening agents include, but are not limited to, aspartame, dextrates, mannitol, saccharin, saccharin calcium, saccharin sodium, sorbitol, sorbitol solution, or mixtures thereof.

Exemplary plasticizers for use with the present invention include, but are not limited to, castor oil, diacetylated monoglycerides, diethyl phthalate, glycerin, mono- and di-acetylated monoglycerides, polyethylene glycol, propylene glycol, and triacetin or mixtures thereof. Suitable viscosity increasing agents include, but are not limited to, acacia, agar, alamic acid, aluminum monostearate, bentonite, bentonite magma, carbomer 934, carboxymethylcellulose calcium, carboxymethylcellulose sodium, carboxymethylcellulose sodium 12, carrageenan, cellulose, microcrystalline cellulose, gelatin, guar gum, hydroxyethyl cellulose, hydroxypropyl cellulose, hydroxypropyl methylcellulose (Nos. 2208; 2906; 2910), magnesium aluminum silicate, methylcellulose, pectin, polyvinyl alcohol, povidone, silica gel, colloidal silicon dioxide, sodium alginate, tragacanth and xanthan gum or mixtures thereof.

Buffering agents that may be used in the present invention include, but are not limited to, magnesium hydroxide, aluminum hydroxide and the like, or mixtures thereof. Examples of humectants include, but are not limited to, glycerol, other humectants or mixtures thereof.

The dosage forms of the present invention may further include one or more of the following: (1) dissolution retarding agents, such as paraffin; (2) absorption accelerators, such as quaternary ammonium compounds; (3) wetting agents, such as, for example, cetyl alcohol and glycerol monostearate; (4) absorbents, such as kaolin and bentonite clay; (5) antioxidants, such as water soluble antioxidants (e.g., ascorbic acid, cysteine hydrochloride, sodium bisulfate, sodium metabisulfate, sodium sulfite and the like), oil soluble antioxidants (e.g., ascorbyl palmitate, hydroxyanisole (BHA), butylated hydroxy toluene (BHT), lecithin, propyl gallate, alpha-tocopherol and the like); and (6) metal chelating agents, such as citric acid, ethylenediamine tetracetic acid (EDTA), sorbitol, tartaric acid, phosphoric acid and the like.

Dosage forms of the present invention, such as a tablet or caplet, may optionally be coated. Inert coating agents typically comprise an inert film-forming agent dispersed in a suitable solvent, and may further comprise other pharmaceutically acceptable adjuvants, such as colorants and plasticizers. Suitable inert coating agents, and methods for coating, are well known in the art, including without limitation aqueous or non-aqueous film coating techniques or microencapsulation. Examples of film-forming or coating agents include, but are not limited to, gelatin, pharmaceutical glaze, shellac, sucrose, titanium dioxide, carnauba wax, microcrystalline wax, celluloses, such as methylcellulose, hydroxymethyl cellulose, carboxymethycellulose, cellulose acetate phthalate, hydroxypropyl methylcellulose (e.g., Nos.: 2208, 2906, 2910), hydroxypropyl cellulose, hydroxypropyl methyl cellulose phthalate (e.g., Nos.: 200731, 220824), hydroxyethylcellulose, methylhydroxyethylcellulose, ethylcellulose which may optionally be cross-linked, and sodium carboxymethyl cellulose; vinyls, such as polyvinyl pyrrolidione, polyvinyl acetate phthalate, glycols, such as polyethylene glycols; acrylics, such as dimethylaminoethyl methacrylate-methacrylate acid ester copolymer, and ethylacrylate-methylmethacrylate copolymer; and other carbohydrate polymers, such as maltodextrins, and polydextrose, or mixtures thereof. The amount of coating agent and the carrier vehicle (aqueous or non-aqueous) used varies upon the type of formulation and mode of administration, and is readily discernible to those of ordinary skill in the art.

A coating of a film forming polymer may optionally be applied to a tablet or caplet (e.g., a capsule shaped tablet) in accordance with the present invention by using one of several types of equipment such as a conventional coating pan, Accelacota, High-Cola or Worster air suspension column. Such equipment typically has an exhaust-system to remove dust and solvent or water vapors to facilitate quick drying. Spray guns or other suitable atomizing equipment may be introduced into the coating pans to provide spray patterns conducive to rapid and uniform coverage of the tablet bed. Normally, heated or cold drying air is introduced over the tablet bed in a continuous or alternate fashion with a spray cycle to expedite drying of the film coating solution.

The coating solution may be sprayed by using positive pneumatic displacement or peristaltic pump systems in a continuous or intermittent spray-dry cycle. The particular type of spray application is selected depending upon the drying efficiency of the coating pan. In most cases, the coating material is sprayed until the tablets are uniformly coated to the desired thickness and the desired appearance of the tablet is achieved. Many different types of coatings may be applied such as enteric, slow release coatings or rapidly dissolving type coatings for fast acting tablets. Preferably, rapidly dissolving type coatings are used to permit more rapid release of the active ingredients, resulting in hastened onset. The thickness of the coating of the film forming polymer applied to a tablet, for example, may vary. However, it is preferred that the thickness simulate the appearance, feel (tactile and mouth feel) and function of a gelatin capsule. Where more rapid or delayed release of the therapeutic agent(s) is desired, one skilled in the art would easily recognize the film type and thickness, if any, to use based on characteristics such as desired blood levels of active ingredient, rate of release, solubility of active ingredient, and desired performance of the dosage form.

A number of suitable film forming agents for use in coating a final dosage form, such as tablets include, for example, methylcellulose, hydroxypropyl methyl cellulose (PHARMACOAT 606 6 cps), polyvinylpyrrolidone (povidone), ethylcellulose (ETHOCEL 10 cps), various derivatives of methacrylic acids and methacrylic acid esters, cellulose acetate phthalate or mixtures thereof.

The method of preparation and the excipients or additives to be incorporated into dosage form (such as a tablet or caplet) are selected in order to give the tablet formulation the desirable physical characteristics while allowing for ease of manufacture (e.g., the rapid compression of tablets). After manufacture, the dose form preferably should have a number of additional attributes, for example, for tablets, such attributes include appearance, hardness, disintegration ability and uniformity, which are influenced both by the method of preparation and by the additives present in the tablet formulation.

Further, it is noted that tablets or other dosage forms of the pharmaceutical compositions of the invention should retain their original size, shape, weight and color under normal handling and storage conditions throughout their shelf life. Thus, for example, excessive powder or solid particles at the bottom of the container, cracks or chips on the face of a tablet, or appearance of crystals on the surface of tablets or on container walls are indicative of physical instability of uncoated tablets. Hence, the effect of mild, uniform and reproducible shaking and tumbling of tablets should be undertaken to insure that the tablets have sufficient physical stability. Tablet hardness can be determined by commercially available hardness testers. In addition, the in vitro availability of the active ingredients should not change appreciably with time.

The tablets, and other dosage forms of the pharmaceutical compositions of the present invention, such as dragees, capsules, pills and granules, may optionally be scored or prepared with coatings and shells, such as enteric coatings and other coatings well known in the pharmaceutical formulating art.

In one embodiment, it is desirable to use a lubricant in pharmaceutical composition and dosage forms of the invention that include an ARB that is poorly soluble or insoluble in water.

5.2.2 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein (i.e., the compounds of this invention) can also be incorporated into the parenteral dosage forms of the invention.

5.2.3 Transdermal, Topical and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers can be used to assist in delivering the active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.2.4 Compositions with Enhanced Stability

The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2^{nd} Ed., Marcel Dekker, New York, N.Y., pp. 379-80 (1995). In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers. Like the amounts and types of excipients, the amounts and specific types of active ingredients in a dosage form may differ depending on factors such as, but not limited to, the route by which it is to be administered to patients.

5.2.5 Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the compounds of this invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.2.6 Kits

In some cases, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a single unit dosage form of the compounds of this invention, or a pharmaceutically acceptable salt, prodrug, solvate, hydrate, clathrate or stereoisomer thereof, and a single unit dosage form of another agent that may be used in combination with the compounds of this invention. Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The invention is further defined by reference to the following non-limiting examples. It will be apparent to those skilled in the art that many modifications, both to materials and methods, can be practiced without departing from the spirit and scope of this invention.

### 6. EXAMPLES

6.1 Preparation of (S)-Amlodipine Salts

The following examples describe the preparation of (S)-amlodipine salts that may be used in the combination as described herein.

A. Formation of (S)-Amlodpine-hemi-D-Tartrate DMAC Solvate from (RS)-Amlodipine Free Base

A solution of D-Tartaric acid (9.48 kg, 63.15 moles) in DMAC (104.2 kg) was added to a slurry of (RS)-Amlodipine free base SEP-174677 (24.9 kg, 60.9 moles) in N,N-Dimethylacetamide (DMAC, 104.3 kg). The reaction mixture was agitated and heated to about 70° C. The reaction mass is held for one hour with agitation at about 70° C. The resulting slurry was then cooled with agitation to about 22° C. over 2.5 to 3 hours with a linear cooling profile at about 0.30° C./min. The slurry was held with agitation at about 22° C. for about 0.5 hr. The solid was isolated by filtration, washed by re-slurrying with DMAC followed by a displacement wash with MTBE. The wet cake was dried at about 45° C. in vacuo to produce (S)-Amlodipine-hemi-D-Tartrate-DMAC solvate (13.92 kg, 24.37 moles, 40.0% yield).

B. Formation of (S)-Amlodipine-L-Malate from (S)-Amlodipine Free Base

A slurry of (S)-Amlodipine free base (19.5 kg, 47.69 moles) in isopropanol-MTBE (141.9 kg IPA, 14.9 kg MTBE, 9.5/1, wt/wt) was agitated and heated to about 50° C. to form a solution. A solution of L-Malic acid (6.68 kg, 49.82 moles) in isopropanol-water (25.45 kg IPA, 5.73 kg water 4.44/1, wt/wt) was then added, and the reaction mixture was held with agitation for about one hour at about 50° C. to form a slurry. The resulting slurry was then cooled with agitation to about 0° C. over 2.5 to 3 hours, with a linear cooling profile at about 0.25° C./min. The slurry was held with agitation at about 0° C. for about one hour. The solid product was isolated by filtration at about 0° C., washed by re-slurrying with cold isopropanol followed by two displacement washes with MTBE. The wet cake was dried at about 60° C. in vacuo to provide (S)-Amlodipine-L-Malate (25.41 kg, 46.79 moles, 98.1% yield).

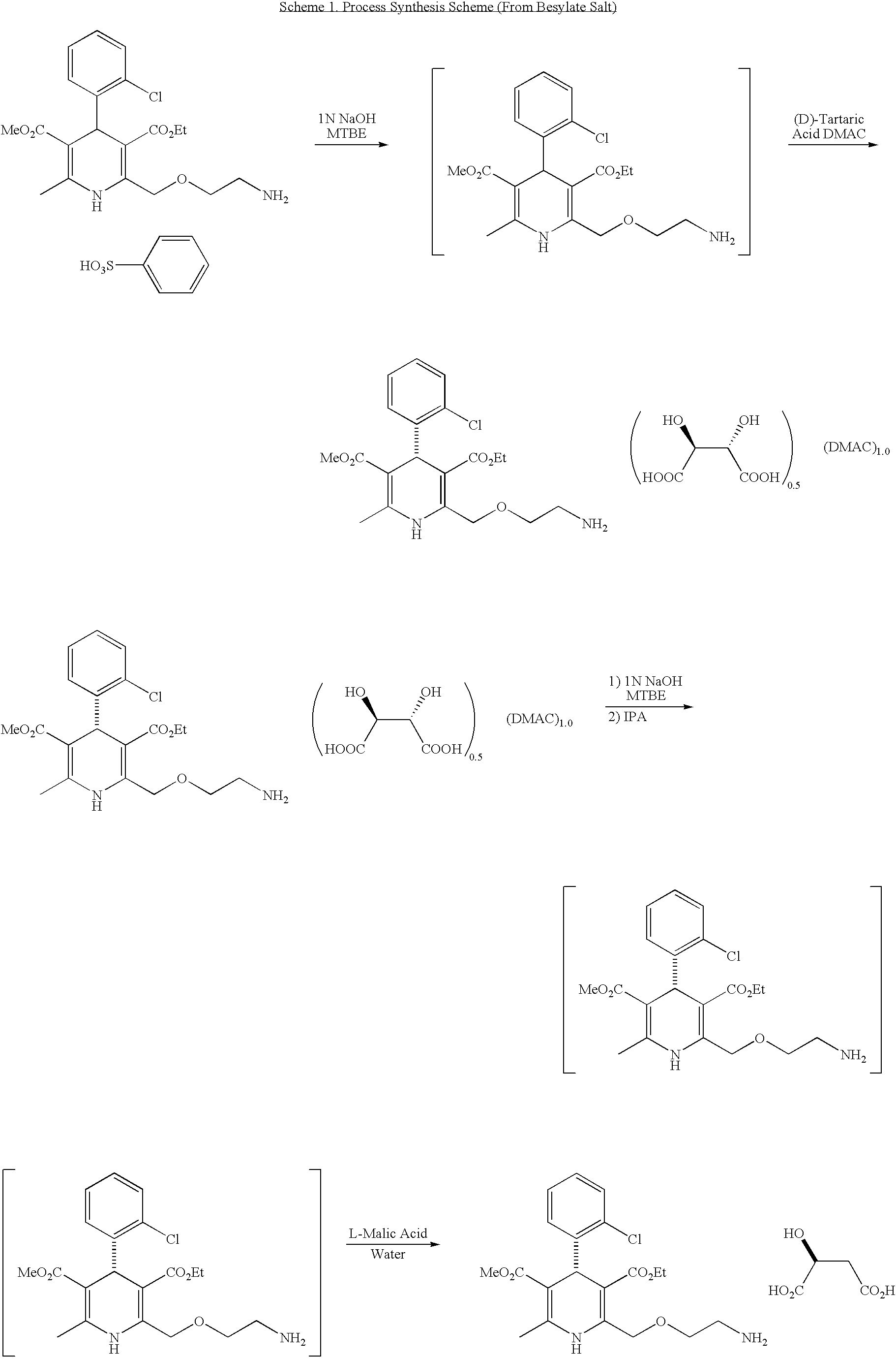

The free base can be used as a starting point in the scheme as well by eliminating the sodium hydroxide/MTBE step and continuing as described.

6.2 Co-Administration of (S)-Amlodipine Malate and ARBs

6.2.1 Study Design

(S)-amlodipine malate and seven ARBs (candasartan, eprosartan, irbesartan, losartan, olmesartan, telmisartan and valsartan) were evaluated. The study was conducted in three parts. Phase I evaluated the time-course of the hypotensive actions of all eight agents ([S]-amlodipine and the seven ARBs). In Phase II, the dose-responsive effects of [S]-amlodipine malate and the seven ARBs were evaluated individually. From this phase of the study, ED₃₀ and ED₅₀ doses of each of the seven ARBs were identified, defined as the dose that produced 30 or 50 percent of the hypotensive effect produced by a rapid i.v. injection (30 μg/kg) of sodium nitroprusside (NTP), a short-acting vasodilator. In Phase III, the dose response to [S]-amlodipine was evaluated in the presence and absence of the ED₃₀ and ED₅₀dose of each of the seven ARBs, with the objective of comparing the extent to which each ARB, at its ED₃₀ or ED₅₀, shifted the dose-response curve of [S]-amlodipine to the left and thus, lowering the apparent ED₅₀ of the calcium channel blocker.

6.2.2 Procedures

Animals were anesthetized with thiobutabarbital sodium (Inactin®, 150 mg/kg, delivered i.p.), tracheotomized and placed on a thermostatically controlled heating pad (37-38° C.). The jugular vein and the carotid artery were cannulated with PE-50 tubing for drug administration and recording of cardiovascular parameters, respectively. Blood pressure was recorded from the carotid artery by means of a pressure transducer and heart rate was derived electronically from the blood pressure signal. The signal output derived electronically from the arterial pressure was analyzed with a digital computer. The software IOX-16 continuously recorded the data every 5 sec during the entire procedure for each rat. Raw arterial pressure (in mmHg) and heart rate (beats per min), as well as changes in these two parameters relative to baseline or pre-drug levels (for doses other than initial dose), both in raw units (mm Hg or beats per min [bpm]) and as percentage change from baseline were captured and analyzed. The animals were allowed to equilibrate for 15-20 min before any treatment.

Doses are expressed in terms of free active substance. Each treatment was administered in a volume of 1 mL/kg by slow (over 20-30 seconds) i.v. bolus for all test articles, in all three phases.

The data were reported as mean±S.E.M. (standard error of the mean) from 3-5 rats per group. Raw arterial pressure (in mmHg) and heart rate (beats per min), as well as changes in these two parameters relative to baseline or pre-drug levels (for doses other than initial dose), both in raw units (mmHg or beats per min [bpm]) and as percentage change from baseline, were captured and analyzed.

ED₃₀ and ED₅₀ for each drug were defined as the dose causing a decline in arterial pressure corresponding to 30% and to 50%, respectively, of the maximal drop in blood pressure produced by fast bolus i.v. injection (30 μg/kg) of sodium nitroprusside.

6.2.3 Phase I

[S]-amlodipine, injected at a dose of 100 μg/kg i.v, caused a fall in blood pressure, with the maximum effect of about 14-17 mmHg decrease in blood pressure reached within 35 to 55 min post-injection. Simultaneous recording of heart rate (HR) in the same animals indicated weak tachycardia.

The time course of the hypotensive effects of the seven ARBs differed, falling into two basic groups. Candasartan and olmesartan, both at doses of 30 μg/kg i.v, demonstrated a longer onset of full activity, with maximum declines of mean blood pressure (MBP) of about 20 and 11 mmHg, respectively, occurring at 30 min post-injection. Attainment of the peak hypotensive effect after administration of eprosartan (100 μg/kg i.v.), losartan (300 μg/kg i.v.), telmisartan (100 μg/kg i.v.) and irbesartan (300 μg/kg i.v.) was observed within 1 min post-injection. Valsartan (100 μg/kg i.v.) exhibited a slightly less rapid timecourse, with a maximum lowering in MBP occurring five minutes after injection. Simultaneous recording of heart rate (HR) in these same animals indicated weak tachycardia within 5 min post-injection of each ARBs.

6.2.4 Phase II

In this phase, the hypotensive response to sodium nitroprusside (NTP, 30 g/kg, i.v. bolus), which was injected once at the end of the equilibration period, was used as a comparator and ED₃₀ and ED₅₀ doses were estimated as the percentage of the maximum NTP effect. NTP induced consistent and reproductive declines in MBP (ranging from 52 to 56% decrease from baseline values) and was without any effect on HR.

(S)-amlodipine malate (3, 10, 30, 100 and 300 μg/kg, i.v.) produced hypotensive responses yielding ED₃₀ and ED₅₀ values of 56 and 110 μg/kg i.v, respectively, with a maximum decline in MBP by 49±1% from baseline values. HR was gradually and concomitantly increased after injection of each dose for both compounds.

Candasartan (3, 10, 30, 100 and 300 μg/kg, i.v.) produced dose-responsive declines in MBP at doses from 3 to 100 μg/kg i.v. The maximum decrease was reached with a dose of 100 μg/kg i.v. (34±2% from baseline values), and there was no further decline with 300 mg/kg i.v. Dose-response curve analysis indicated an estimated ED₃₀ value of 8 μg/kg i.v., and an estimated ED₅₀ value of 52 μg/kg i.v.

Olmesartan (3, 10, 30, 100 and 300 μg/kg, i.v.) produced dose-related falls in MBP from 3 to 300 μg/kg i.v. The maximum decrease was reached with dose of 300 μg/kg i.v. (32±5% from baseline values). Dose-response curve analysis indicated an estimated ED₃₀ value of 13 μg/kg i.v., and an estimated ED₅₀ value of 103 μg/kg i.v.

Losartan (30, 100, 300, 1000 and 3000 μg/kg, i.v.) produced weak dose-related falls in MBP from 30 to 3000 μg/kg i.v. The maximum decrease was reached with dose of 3,000 μg/kg i.v. (23±3% from baseline values). Dose-response curve analysis indicated an estimated ED₃₀ value of 625 μg/kg i.v. and an extrapolated ED₅₀ value of 10,000 μg/kg i.v.

Irbesartan (30, 100, 300, 1000 and 3000 μg/kg, i.v.) produced dose-related falls in MBP from 30 to 3000 μg/kg i.v. The maximum decrease was reached with dose of 3,000 μg/kg i.v. (28±1% from baseline values). Dose-response curve analysis indicated an estimated ED₃₀ value of 649 μg/kg i.v., and an estimated ED₅₀ value of 2,211 μg/kg i.v.

Telmisartan (10, 30, 100, 300 and 1000 μg/kg, i.v.) produced weak dose-related falls in MBP from 10 to 300 μg/kg i.v. The maximum decrease was reached with dose of 300 μg/kg i.v. (19±2% from baseline values), and there was no further fall with 1000 mg/kg i.v. (23±3% from baseline values). Dose-response curve analysis indicated an estimated ED₃₀ value of 242 μg/kg i.v., and an extrapolated ED₅₀ value of ˜2,000 μg/kg i.v.

Valsartan (10, 30, 100, 300 and 1000 μg/kg, i.v.) produced weak dose-related falls in MBP from 10 to 300 μg/kg i.v. The maximum decrease was reached with dose of 300 μg/kg i.v. (18±3% from baseline values), and there was no further fall with 1000 mg/kg, i.v. (19±3% from baseline values). Dose-response curve analysis indicated an estimated ED₃₀ value of 229 μg/kg i.v., and an extrapolated ED₅₀ value of ˜4,000 μg/kg i.v.

Eprosartan (10, 30, 100, 300, 1000 and 3000 μg/kg, i.v.) produced weak dose-related falls in MBP from 10 to 1000 μg/kg i.v. The maximum decrease was reached with dose of 1000 μg/kg i.v. (22±2% from baseline values), and there was no further fall with 3000 mg/kg i.v. (23±2% from baseline values). Dose-response curve analysis indicated an estimated ED₃₀ value of 104 μg/kg i.v., and an extrapolated ED₅₀ value of ˜7,000 μg/kg i.v.

ED₃₀ and ED₅₀ doses (μg/kg i.v.) derived from dose response curves for (S)-amlodipine malate and the seven ARBs are shown in the table below. Because several of the ARBs did not produce a hypotensive effect that reached 50% of the maximum NTP effect, the ED₅₀ values were extrapolated from dose response curves. An ED₃₀ value could be derived for all seven ARBs by interpolation of each one's dose-response curve.

TABLE 1
ED30 and ED50 Values of (S)-amlodipine malate and ARBs

| ARB's | ED₃₀ (μg/kg) | ED₅₀ (μg/kg) |
|---|---|---|
| (S)-Amlodipine | ND | 110 |
| Valsartan | 229 | ˜4,000-10,000 |
| Losartan | 625 | 10,000 |
| Candasartan | 8 | 52 |
| Telmisartan | 242 | ˜2,000-1,100 |
| Olmesartan | 13 | 103 |
| Eprosartan | 104 | ˜7,000-10,000 |
| Irbesartan | 649 | 2,211 |

6.2.5 Phase III

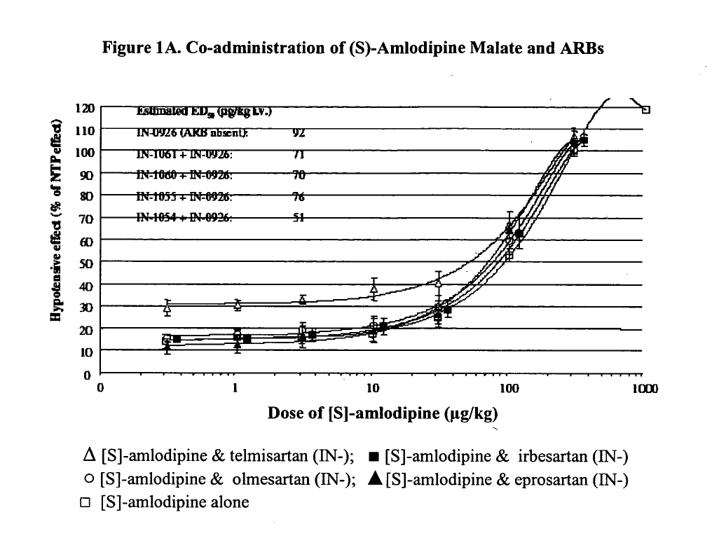

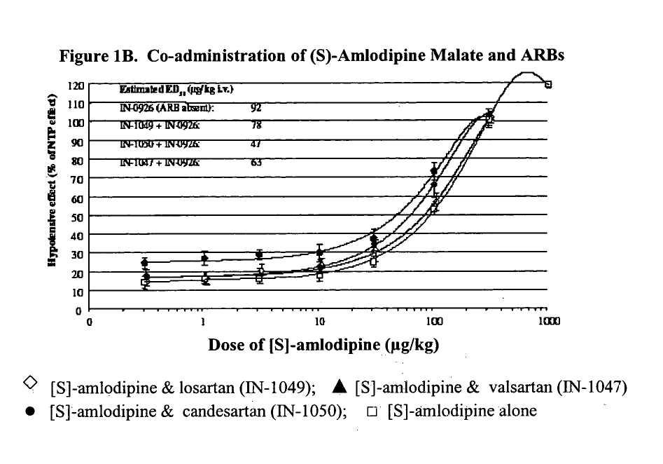

(S)-amlodipine malate, when evaluated in the absence of an ARB, produced a dose-response curve and resultant ED₅₀ value (92 μg/kg) nearly identical to what was obtained in Phase II, (110 μg/kg i.v.). Dose-response curves with (S)-amlodipine malate in the absence and presence of each of the seven ARBs at its ED₃₀ are shown in FIGS. 1A and 1B. The data shown in the figures are from experiments in which ED₃₀ doses of each of the ARBs was combined with (S)-amlodipine malate. These were chosen because ED₃₀ doses were obtainable by direct interpolation of dose response curves from Phase II. For several of the ARBs, doses corresponding to ED₅₀ were obtained from extrapolation of their dose response curves.

Because the dose response curves for telmisartan plus (S)-amlodipine malate (open triangles in FIG. 1A) and candesartan plus (S)-amlodipine malate (solid circles in FIG. 1B) were offset (i.e., lying above the dose response curve for (S)-amlodipine malate alone), these two were excluded from consideration of exerting a leftward shift of (S)-amlodipine malate's dose response curve. Of the remaining five ARBs, valsartan produced the greatest leftward shift to the left of (S)-amlodipine malate's dose-response curve, yielding a new “apparent ED₅₀” of 63 μg/kg. The remaining ARBs produced the following apparent ED₅₀s in (S)-amlodipine malate when combined with the calcium channel blocker: eprosartan (70 μg/kg); irbesartan (71 μg/kg); olmesartan (76 μg/kg); and losartan (78 μg/kg). These are presented in Table 2, below, along with an “adjustment factor,” which represents the extent to which one might predict that the extent to which (S)-amlodipine malate could be lowered, and still enjoy the same anti-hypertensive effect in vivo. Because the untoward effects of amlodipine, particularly the edema-promoting properties, it is conceivable that the concomitant administration of one of the ARBs below, most notably valsartan, at dose that would be well-tolerated, would provide more than adequate anti-hypertensive actions, but with a lower incidence of edema.

TABLE 2
Effects of Co-administration of (S)-Amlodipine Malate and ARBs

| | | Potential Dosage |
|---|---|---|
| | ED₅₀ (μg/kg) | Adjustment (%) |
| No ARB | 92 | — |
| ([S]-amlodipine alone) | | |
| Valsartan | 63 | 32 |
| Losartan | 78 | 15 |
| Candasartan | ND | ND |
| Telmisartan | ND | ND |
| Olmesartan | 76 | 17 |
| Eprosartan | 70 | 24 |
| Irbesartan | 71 | 23 |

6.3 Oral Formulation: Capsules

Followings are exemplary capsule formulations of compositions of this invention.

TABLE 3
(S)-Amlodipine and Irbesartan

| | Quantity per Capsule in mg | | | | | |
|---|---|---|---|---|---|---|
| Ingredients | A | B | C | D | E | F |

| | | | | | | |
|---|---|---|---|---|---|---|
| (S)-Amlodipine | 0.5 | 2.5 | 5.0 | 0.5 | 2.5 | 5.0 |
| Irbesartan | 50.0 | 50.0 | 50.0 | 100.0 | 100.0 | 100.0 |
| Lactose | 133.5 | 131.5 | 129.0 | 83.5 | 81.5 | 79.0 |
| Corn Starch | 15.0 | 15.0 | 15.0 | 15.0 | 15.0 | 15.0 |
| Magnesium Stearate | 1.0 | 1.0 | 1.0 | 1.0 | 1.0 | 1.0 |
| Total Weight | 200.0 | 200.0 | 200.0 | 200.0 | 200.0 | 200.0 |

(S)-amlodipine, irbesartan, lactose and corn starch are blended until uniform; then the magnesium stearate is blended into the resulting powder. The resulting powder is encapsulated into suitably sized two-piece hard gelatin capsules.

TABLE 4
(S)-Amlodipine and Olmesartan

| | Quantity per Capsule in mg | | | | | |
|---|---|---|---|---|---|---|
| Ingredients | A | B | C | D | E | F |
| (S)-Amlodipine | 0.5 | 2.5 | 5.0 | 0.5 | 2.5 | 5.0 |
| Olmesartan | 5.0 | 5.0 | 5.0 | 20.0 | 20.0 | 20.0 |
| Lactose | 78.5 | 77.5 | 75.0 | 63.5 | 61.5 | 59.0 |
| Corn Starch | 15.0 | 15.0 | 15.0 | 15.0 | 15.0 | 15.0 |
| Magnesium Stearate | 1.0 | 1.0 | 1.0 | 1.0 | 1.0 | 1.0 |
| Total Weight | 100.0 | 100.0 | 100.0 | 100.0 | 100.0 | 100.0 |

(S)-amlodipine, olmesartan, lactose and corn starch are blended until uniform; then the magnesium stearate is blended into the resulting powder. The resulting powder is encapsulated into suitably sized two-piece hard gelatin capsules.

TABLE 5
(S)-Amlodipine and Telmisartan

| | Quantity per Capsule in mg | | | | | |
|---|---|---|---|---|---|---|
| Ingredients | A | B | C | D | E | F |
| (S)-Amlodipine | 0.5 | 2.5 | 5.0 | 0.5 | 2.5 | 5.0 |
| Telmisartan | 10.0 | 10.0 | 10.0 | 20.0 | 20.0 | 20.0 |
| Lactose | 73.5 | 71.5 | 69.0 | 63.5 | 61.5 | 59.0 |
| Corn Starch | 15.0 | 15.0 | 15.0 | 15.0 | 15.0 | 15.0 |
| Magnesium Stearate | 1.0 | 1.0 | 1.0 | 1.0 | 1.0 | 1.0 |
| Total Weight | 100.0 | 100.0 | 100.0 | 100.0 | 100.0 | 100.0 |

(S)-amlodipine, telmisartan, lactose and corn starch are blended until uniform; then the magnesium stearate is blended into the resulting powder. The resulting powder is encapsulated into suitably sized two-piece hard gelatin capsules.

TABLE 6
(S)-Amlodipine and Eprosartan

| | Quantity per Capsule in mg | | | | | |
|---|---|---|---|---|---|---|
| Ingredients | A | B | C | D | E | F |
| (S)-Amlodipine | 0.5 | 2.5 | 5.0 | 0.5 | 2.5 | 5.0 |
| Eprosartan | 100.0 | 100.0 | 100.0 | 200.0 | 200.0 | 200.0 |
| Lactose | 83.5 | 82.5 | 80.0 | 83.5 | 82.5 | 80.0 |
| Corn Starch | 15.0 | 15.0 | 15.0 | 15.0 | 15.0 | 15.0 |
| Magnesium Stearate | 1.0 | 1.0 | 1.0 | 1.0 | 1.0 | 1.0 |
| Total Weight | 200.0 | 200.0 | 200.0 | 300.0 | 300.0 | 300.0 |

(S)-amlodipine, eprosartan, lactose and corn starch are blended until uniform; then the magnesium stearate is blended into the resulting powder. The resulting powder is encapsulated into suitably sized two-piece hard gelatin capsules.

6.4 Oral Formulation: Tablets

S-amlodipine and losartan tablets may be prepared according to this example.

(S)-Amlodipine & Losartan Potassium Tablets
Description: 7/16″ Film-coated flat faced beveled edge
compressed tablet; Table weight 310 mg
Batch size: 310 grams (˜1000 tablets)

| | | | | |
|---|---|---|---|---|
| | | | mg/ | Composition |
| Item | Component | Source | tablet* | (% w/w) |

| 1 | (S)-Amlodipine(L) - | Sepracor Inc. | 6.64 | 2.14 |
|---|---|---|---|---|
| | Malate (SEP-4675-15) | | (5.0)● | |
| 2 | Losartan Potassium | TAIZHOU | 100.0 | 32.26 |
| 3 | Microcrystalline | FMC | 135.36 | 43.66 |
| | Cellulose, NF | Corporation | | |
| | (Avicel PH 302) | | | |
| 4 | Pregelatinized Starch, | Colorcon | 41.50 | 13.39 |
| | NF | | | |
| | (Starch 1500) | | | |
| 5 | Sodium Starch | FMC | 15.00 | 4.84 |
| | Glycolate, NF | Corporation | | |
| | (Explotab) | | | |
| 6 | Magnesium Stearate, | Mallinckrodt | 1.50 | 0.48 |
| | NF | | | |
| 7 | Opadry II | Colorcon | 10 | 3.23 |

| | | | | |
|---|---|---|---|---|
| 8 | Water | — | — | qs |

| Total | 310 | mg | 100 |
|---|---|---|---|
| *Target Quantity | | | |
| ●Free base | | | |
| (S)-amlodipine-(L)-malate, microcrystalline cellulose, NF (Avicel PH 302), pregelatinized starch, NF (Starch 1500) and sodium starch glycolate, NF (Explotab) were blended in twin shell blender for 5 minutes. Losartan potassium was added to this mixture, and the mixture was blended for 20 minutes. Then, magnesium stearate was added to the | | | |
| # mixture, and the resulting mixture was further blended for 3 minutes. The final blend was compressed on conventional tablet press. Coating suspension was prepared from Opadry II and water, and the compressed tablets were coated in 24″ CompuLab. | | | |

The embodiments of the invention described above are intended to be merely exemplary and those skilled in the art will recognize, or be able to ascertain using no more than routine experimentation, numerous equivalents to the specific procedures described herein. All such equivalents are considered to be within the scope of the invention and are encompassed by the following claims.

All of the patents, patent applications and publications referred to herein are incorporated in their entireties. Moreover, citation or identification of any reference in this application is not an admission that such reference is available as prior art to this invention. The full scope of the invention is better understood with reference to the appended claims.

1. A method of treating or managing a cardiovascular disease or disorder comprising administering to a patient in need of such treatment a therapeutically effective amount of:
enantiomerically pure (S)-amlodipine, or a pharmaceutically acceptable prodrug, salt, or solvate thereof; and
an angiotensin receptor blocker, or a pharmaceutically acceptable prodrug, salt or solvate thereof;
wherein the angiotensin receptor blocker is candesartan, eprosartan, telmisartan, irbesartan, pratosartan, valsartan or olmesartan.

2. A method of preventing a cardiovascular disease or disorder comprising administering to a patient in need of such prevention a prophylactically effective amount of:
enantiomerically pure (S)-amlodipine, or a pharmaceutically acceptable prodrug, salt, or solvate thereof; and
an angiotensin receptor blocker, or a pharmaceutically acceptable prodrug, salt or solvate thereof;
wherein the angiotensin receptor blocker is candesartan, eprosartan, telmisartan, irbesartan, pratosartan, valsartan or olmesartan.

3. A method of treating or managing a cardiovascular disease or disorder comprising administering to a patient in need of such treatment a therapeutically effective amount of:
enantiomerically pure (S)-amlodipine, or a pharmaceutically acceptable prodrug, salt, or solvate thereof;
an angiotensin receptor blocker, or a pharmaceutically acceptable prodrug, salt or solvate thereof, wherein the angiotensin receptor blocker is candesartan, eprosartan, telmisartan, irbesartan, pratosartan, valsartan or olmesartan; and
a diuretic agent.

4. A method of preventing a cardiovascular disease or disorder comprising administering to a patient in need of such prevention a prophylactically effective amount of:
enantiomerically pure (S)-amlodipine, or a pharmaceutically acceptable prodrug, salt, or solvate thereof;
an angiotensin receptor blocker, or a pharmaceutically acceptable prodrug, salt or solvate thereof wherein the angiotensin receptor blocker is candesartan, eprosartan, telmisartan, irbesartan, pratosartan, valsartan or olmesartan; and
a diuretic agent.

5. The method of claim 1, 2, 3, or 4, wherein the angiotensin receptor blocker is valsartan.

6. The method of claim 1, 2, 3, or 4, wherein the angiotensin receptor blocker is eprosartan.

7. The method of claim 1, 2, 3, or 4, wherein the angiotensin receptor blocker is irbesartan.

8. The method of claim 1, 2, 3, or 4, wherein the angiotensin receptor blocker is telmisartan.

9. The method of claim 1, 2, 3, or 4, wherein the angiotensin receptor blocker is candesartan, pratosartan, or olmesartan.

10. The method of claim 1, 2, 3, or 4, wherein the patient is a human.

11. The method of claim 1, 2, 3, or 4, wherein the disease or disorder is acute or chronic renal failure, angina pectoris, arterial spasm, cardiac arrhythmias, cardiac hypertrophy, cerebral ischemia, congestive heart failure, coronary myocardial infarction, hypertension, ischemia reperfusion injury, ischemic myocardial necrosis, stroke, Raynaud's phenomenon, diabetic nephropathy, or renal impairment.

12. The method of claim 11, wherein the disease or disorder is hypertension.

13. The method of claim 11, wherein the disease or disorder is angina.

14. The method of claim 1, 2, 3, or 4, wherein the (S)-amlodipine, or a pharmaceutically acceptable prodrug, salt, or solvate thereof, is administered in an amount of from about 0.01 mg to about 100 mg per day.

15. The method of claim 14 wherein the (S)-amlodipine, or a pharmaceutically acceptable prodrug, salt, or solvate thereof, is administered in an amount of from about 1 mg to about 10 mg per day.

16. The method of claim 3 or 4, wherein the diuretic agent is acetazolamide, dichlorphenamide, methazolamide glycerin, isosorbide, mannitol, urea, furosemide, bumetanide, azosemide, piretanide, ethacrynic acid, muzolimine, torsemide, tripamide, bendroflumethiazide, chlorothiazide, hydrochlorothiazide, dihydrochlorothiazide, hydroflumethiazide, methylchlorthiazide, polythiazide, trichlormethiazide, chlorthalidone, indapamide, metolazone, quinethazone; amiloride, triameterene, spironolactone, conivaptan, mozavaptan, or eplerenone.

17. A pharmaceutical composition comprising:
enantiomerically pure (S)-amlodipine, or a pharmaceutically acceptable prodrug, salt, or solvate thereof, and
an angiotensin receptor blocker, or a pharmaceutically acceptable prodrug, salt or solvate thereof;
wherein the angiotensin receptor blocker is candesartan, eprosartan, telmisartan, irbesartan, pratosartan, valsartan or olmesartan.

18. A pharmaceutical composition comprising:
enantiomerically pure (S)-amlodipine, or a pharmaceutically acceptable prodrug, salt, or solvate thereof,
an angiotensin receptor blocker, or a pharmaceutically acceptable prodrug, salt or solvate thereof, wherein the angiotensin receptor blocker is candesartan, eprosartan, telmisartan, irbesartan, pratosartan, valsartan or olmesartan; and
a diuretic agent.

19. The pharmaceutical composition of claim 18, wherein the diuretic agent is acetazolamide, dichlorphenamide, methazolamide glycerin, isosorbide, mannitol, urea, furosemide, bumetanide, azosemide, piretanide, ethacrynic acid, muzolimine, torsemide, tripamide, bendroflumethiazide, chlorothiazide, hydrochlorothiazide, dihydrochlorothiazide, hydroflumethiazide, methylchlorthiazide, polythiazide, trichlormethiazide, chlorthalidone, indapamide, metolazone, quinethazone; amiloride, triameterene, spironolactone, conivaptan, mozavaptan, or eplerenone.

20. The pharmaceutical composition of claim 17 or 18, wherein the angiotensin receptor blocker is valsartan.

21. The pharmaceutical composition of claim 17 or 18, wherein the angiotensin receptor blocker is eprosartan.

22. The pharmaceutical composition of claim 17 or 18, wherein the angiotensin receptor blocker is irbesartan.

23. The pharmaceutical composition of claim 17 or 18, wherein the angiotensin receptor blocker is telmisartan.

24. The pharmaceutical composition of claim 17 or 18, wherein the angiotensin receptor blocker is candesartan, pratosartan, or olmesartan.

25. The pharmaceutical composition of claim 17 or 18, which further comprises one or more pharmaceutically acceptable excipients.

26. The pharmaceutical composition of claim 25, wherein said excipient is a surfactant.

27. A single unit dosage form comprising:
enantiomerically pure (S)-amlodipine, or a pharmaceutically acceptable prodrug, salt, or solvate thereof; and
an angiotensin receptor blocker, or a pharmaceutically acceptable prodrug, salt or solvate thereof, wherein the angiotensin receptor blocker is candesartan, eprosartan, telmisartan, irbesartan, pratosartan, valsartan or olmesartan.

28. A single unit dosage form comprising:
enantiomerically pure (S)-amlodipine, or a pharmaceutically acceptable prodrug, salt, or solvate thereof;
an angiotensin receptor blocker, or a pharmaceutically acceptable prodrug, salt or solvate thereof;
wherein the angiotensin receptor blocker is candesartan, eprosartan, telmisartan, losartan, irbesartan, pratosartan, valsartan or olmesartan; and a diuretic agent.

29. The dosage form of claim 28, wherein the diuretic agent is acetazolamide, dichlorphenamide, methazolamide glycerin, isosorbide, mannitol, urea, furosemide, bumetanide, azosemide, piretanide, ethacrynic acid, muzolimine, torsemide, tripamide, bendroflumethiazide, chlorothiazide, hydrochlorothiazide, dihydrochlorothiazide, hydroflumethiazide, methylchlorthiazide, polythiazide, trichlormethiazide, chlorthalidone, indapamide, metolazone, quinethazone; amiloride, triameterene, spironolactone, conivaptan, mozavaptan, or eplerenone.

30. The dosage form of claim 27 or 28, wherein the angiotensin receptor blocker is valsartan.

31. The dosage form of claim 27 or 28, wherein the angiotensin receptor blocker is eprosartan.

32. The dosage form of claim 27 or 28, wherein the angiotensin receptor blocker is irbesartan.

33. The dosage form of claim 27 or 28, wherein the angiotensin receptor blocker is telmisartan.

34. The dosage form of claim 27 or 28, wherein the angiotensin receptor blocker is candesartan, pratosartan, or olmesartan.

35. The dosage form of claim 27 or 28, wherein the dosage from is suitable for oral or parenteral administration.

36. A method treating, preventing, or managing a cardiovascular disease or disorder comprising administering to a patient in need of such treatment a pharmaceutical composition of claim 17 or 18.

37. A method of treating, preventing or managing a cardiovascular disease or disorder comprising administering to a patient in need of such treatment a single unit dosage form of claim 27 or 28.

38. The method of claim 36, wherein the cardiovascular disease or disorder is hypertension.

39. The method of claim 37, wherein the cardiovascular disease or disorder is hypertension.

40. The method of claim 36, wherein the cardiovascular disease or disorder is angina.

41. The method of claim 37, wherein the cardiovascular disease or disorder is angina.
